(19) United States
(12) Patent Application Publication
(10) Pub. No.: US 2009/0143328 A1
(43) Pub. Date: Jun. 4, 2009

(54) Method of Treating Cancer by Administration of Topical Active Corticosteroids

(76) Inventor: George McDonald, Bellvue, WA (US)

Correspondence Address: CATALYST LAW GROUP, APC, 9710 SCRANTON ROAD, SUITE S-170, SAN DIEGO, CA, 92121, US

(21) Appl. No.: 12/186,492

(22) Filed: Aug. 5, 2008

Related U.S. Application Data
(63) Continuation-in-part of application No. 09/928,890, filed on Aug. 13, 2001, now abandoned.

(51) Int. Cl. A61K 31/575; A61K 31/52; A61P 35/00; A61P 35/02

(52) U.S. Cl. 514/48; 514/181; 514/171; 514/170

(57) ABSTRACT

The present invention provides for methods of treating cancer comprising administering a topical active corticosteroid in conjunction with a form of non-myeloablative conditioning, wherein the above regimen results in a reduction or elimination of cancer cells in an individual.

### RELATED APPLICATIONS

This application is a continuation-in-part application of U.S. application Ser. No. 09/928,890, filed on Aug. 13, 2001.

### FIELD OF THE INVENTION

This invention relates to methods useful for the treatment of cancer. More particularly, this invention relates to methods that may be used in controlling a graft-versus-leukemia (GVL) reaction in an individual.

### BACKGROUND OF THE INVENTION

Leukemia, lymphoma and myeloma are cancers that originate in the bone marrow (in the case of leukemia and myeloma) or in lymphatic tissues (in the case of lymphoma). Leukemia, lymphoma and myeloma are considered to be related cancers, because they involve the uncontrolled growth of cells having similar functions and origins. The diseases result from an acquired (i.e., not inherited) genetic injury to the DNA of a single cell, which becomes abnormal (malignant) and multiplies continuously. The accumulation of malignant cells interferes with the body's production of healthy blood cells and makes the body unable to protect itself against infections.

Treatment of leukemia, lymphoma and myeloma usually involves one or more forms of chemotherapy and/or radiation therapy. These treatments destroy the malignant cells, but also destroy the body's healthy blood cells as well. Allogeneic bone marrow transplantation (BMT) is an effective therapy useful in the treatment of many hematologic malignancies. In allogeneic BMT, bone marrow (or, in some cases, peripheral blood) from an unrelated or a related (but not identical twin) donor is used to replace the healthy blood cells in the cancer patient. The bone marrow (or peripheral blood) contains stem cells, which are the precursors to all the different cell types (e.g., red cells, phagocytes, platelets and lymphocytes) found in blood. Allogeneic BMT has both a restorative effect and a curative effect. The. restorative effect arises from the ability of the stem cells to repopulate the cellular components of blood. The curative properties of allogeneic BMT derive largely from a graft-versus-leukemia (GVL) effect.

The hematopoietic cells from the donor (specifically, the T lymphocytes) attack the cancerous cells, enhancing the suppressive effects of the other forms of treatment. Essentially, the GVL effect comprises an attack on the residual tumor cells by the blood cells derived from the BMT, making it less likely that the malignancy will return after transplant. Controlling the GVL effect prevents escalation of the GVL effect into other worsening conditions, such as graft versus host disease (GVHD).

Allogeneic haematopoietic stem-cell transplantation was developed as a strategy to prevent the bone-marrow toxicity that is caused by intensive chemoradiotherapy regimens. This approach cures a significant percentage of patients who have otherwise fatal hematological malignancies. Reciprocal immune reactions between donor and recipient are a principal feature of allogeneic stem-cell transplantation, and have both deleterious and beneficial consequences. Key to these immune reactions are human leukocyte antigen (HLA) class I and II molecules, which are expressed on the cell surface and present peptides for recognition by CD8+ and CD4+ T cells, respectively. T cells in the graft can react against recipient HLA-peptide complexes, leading to GVHD in the skin, gastrointestinal tract and/or liver. Less frequently, residual T cells in the host react against donor stem cells, leading to graft rejection. The highest risk of GVHD and graft rejection occurs in transplants between HLA-mismatched individuals. However, unless donor T cells are depleted from the stem-cell graft, GVHD also frequently occurs after HLA-matched stem-cell transplantation because of recognition of minor histocompatibility antigens, which are polymorphic peptides that are displayed by HLA molecules of recipient cells. The ability of allogeneic bone-marrow cells and peripheral-blood stem cells to cure leukaemia remains the most striking example of the ability of the human immune system to recognize and destroy tumors. However, harnessing this GVL effect to improve outcome for patients with advanced disease and segregating it from GVHD have proven to be key challenges (See Bleakely et al., Molecules and Mechanisms of the Graft-Versus-Leukemia Effect, Nat. Rev. Cancer 4(5) :371-380, (2004)).

Animal models and human studies of allogeneic stem-cell transplantation show that immunological non-identity between donor and recipient is also responsible for a GVL effect that leads to tumor eradication (Barnes et al., Treatment of murine leukemia with x-rays and homologous bone marrow, Br. Med. J. 32, 626-627 (1956); Weiden et al., Antileukemic effect of graft-versus-host disease in human recipients of allogeneic-marrow grafts, N. Engl. J. Med. 300, 1068-1073 (1979)). In humans, recipients of allogeneic stem-cell transplants were found to have a lower risk of leukemic relapse than recipients of syngeneic stem-cell transplants or recipients of T-cell-depleted allogeneic stem-cell transplants (Horowitz et al., Graft-versus-leukemia reactions after bone marrow transplantation, Blood 75, 555-562 (1990); Marmont et al., T-cell depletion of HLA-identical transplants in leukemia, Blood 78, 2120-2130 (1991)). The GVL effect, is greatest in the subset of allogeneic-stem-cell-transplant recipients with GVHD, but the risk of relapse is also reduced in patients without GVHD (Passweg et al., Graft-versus-leukemia effects in T lineage and B lineage acute lymphoblastic leukemia, Bone Marrow Transplant. 21, 153-158 (1998)). The potency of the GVL effect is illustrated by the use of donor-lymphocyte infusion to treat patients with leukemia who experience a relapse after receiving a transplant. Remarkably, donor-lymphocyte infusion can induce a durable remission in most patients with chronic myelogenous leukemia (CML) and in some patients with acute leukemia (Kolb et al., Graft-versus-leukemia effect of donor lymphocyte transfusions in marrow grafted patients, Blood 86, 2041-2050 (1995); Collins et al., Donor leukocyte infusions in 140 patients with relapsed malignancy after allogeneic bone marrow transplantation, J. Clin. Oncol. 15, 433-444 (1997)).

U.S. Pat. No. 6,096,731 (McDonald) describes a method for the treatment of GVHD that comprises administration of a prophylactically effective amount of a topically active corticosteroid (TAC) to a patient following intestinal or liver transplantation. The TAC is administered for a period of time effective to prior to presentation of symptoms associated with GVHD. However, no information was given relating to methods of treatment of cancer by controlling a GVL reaction.

While significant advances have been made with regard to the treatment of GVHD following bone marrow transplantation, there is still a need in the art for improved methods for the treatment of certain cancers by controlling the GVL effect and preventing the damage associated with a variety of blood borne cancers.

### BRIEF DESCRIPTION OF THE DRAWINGS

FIG. 1 depicts a graph indicating time to treatment failure through study Day 50 estimates based on Kaplan-Meier method (All randomized subjects). The p-value is based on the stratified log-rank test (Significance level of 0.05 (two-sided)).

FIG. 2 depicts a graph showing time to treatment failure through study Day 80. Estimates based on Kaplan-Meier method (All randomized subjects). P-value is based on the stratified log-rank test. (Significance level of 0.05 (two-sided)).

FIG. 3 depicts a graph indicating duration of overall survival post-randomization (Safety population). P-value is based on the log-rank test with a significance level of 0.05 (two-sided).

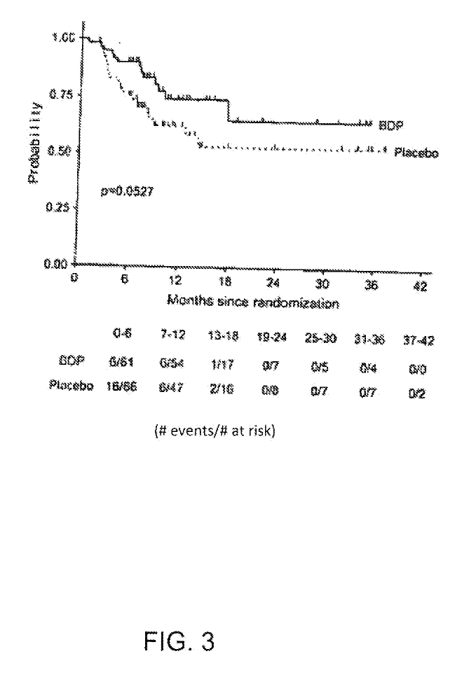

### SUMMARY OF THE INVENTION

The present invention discloses a method for the improved, treatment of blood borne cancers, such as lymphomas, leukemia, and myeloma. The method comprises the oral administration of an effective amount of a TAC to a patient, in conjunction with non-myeloablative conditioning, who has undergone allogeneic hematopoietic cell transplantation, in order to provide a reduction or elimination of tumors. Administration of the TAC controls a GVL reaction that is induced following an allogeneic hematopoietic cell transplantation and the TAC, together with non-myeloablative conditioning, provides the therapeutic benefit of decreasing or eliminating tumors. The GVL reaction effects killing of cancerous tumor cells in the blood, mediated by the cells derived from the allogeneic hematopoietic cell transplantation.

One aspect of the present invention comprises a method of treating an animal with cancer who has received an allogeneic hematopoietic cell transplant, comprising administering to the animal an amount of an oral TAC in conjunction with a form of non-myeloablative conditioning, the TAC and conditioning effective to reduce or eliminate the number of cancer cells in the blood of the animal.

The above and other objects, features and advantages of the present invention will become apparent from the following description.

### DETAILED DESCRIPTION OF THE INVENTION

The present invention is directed to a method for the treatment of cancer by controlling a GVL reaction following allogeneic hematopoietic cell transplantation. The method comprises the oral administration of an effective amount of a TAC and non-myeloblative conditioning to a patient who has undergone, or immediately prior to undergoing, allogeneic hematopoietic cell transplantation.

As used herein, “hematopoietic cell transplantation” refers to bone marrow transplantation, peripheral blood stem cell transplantation, umbilical vein blood transplantation, or any other source of pleuripotent hematopoietic stem cells.

The term “effective amount” refers to an amount of the TAC that reduces or eliminates the number of cancer cells in the blood of a cancer patient. Alternatively, the term refers to a form of non-myeloablative conditioning to be used in conjunction with the TAC administration.

As used herein, “non-myeloablative conditioning” refers to regimens which use significantly lower doses of pre-transplant chemotherapy drugs and/or radiation than the traditional high-dose, myeloablative regimens. These non-myelobalative regimens typically use combinations of chemotherapy drugs including, but not limited to, fludarabine, busulfan, ATG and melphalan, with or without low-dose radiation.

As used herein, the term “treatment” means administration of a therapy effective to augment or maintain a GVL reaction in an individual having a form of cancer.

The term “patient” refers to any animal that may develop cancer, and will most often refer to a human.

Patients who may benefit from the methods of the present invention include those who have undergone or will undergo allogeneic hematopoietic cell or organ allograft transplantation; those who are or will be allogenic hematopoietic cell recipients who have typically received marrow-ablative chemotherapy and/or total body irradiation followed by donor hematopoietic cell infusion; or patients who have undergone or will undergo intestinal or liver organ transplantation. Such procedures are well known to those skilled in this field, and the steps employed in these procedures do not form an element of the present invention.

An important aspect of the present invention is that the TAC is orally administered such that it is topically administered to the intestinal and/or liver tissue. Thus, oral administration, as that term is used herein, is intended to exclude any form of systemic administration, such as by intravenous injection. Oral administration ensures that the TAC has little systemic availability, but high topical activity on intestinal and/or liver tissue. Such limited distribution results in fewer side effects, which is a significant advantage of this invention.

The recognition of the GVL effect is now driving the evolution of allogeneic stem-cell transplantation towards an immunotherapeutic approach that does not require toxic chemoradiotherapy for tumor eradication. Animal experiments have shown that a less intensive approach, known as non-myeloablative conditioning, can suppress recipient immunity sufficiently to allow allogeneic stem- and immune-cell engraftment. Clinical trials are now using non-myeloablative regimens consisting of fludarabine and low-dose chemotherapy or total-body irradiation. These usually achieve donor-cell engraftment with a decrease in both organ toxicity and early mortality, compared with myeloablative regimens. Non-myeloablative conditioning makes it possible to perform bone-marrow transplantation safely in older patients and those with compromised organ function, but provides minimal direct antitumor activity. The lack of significant antitumor activity of these conditioning regimens means that tumor eradication relies almost exclusively on the GVL effect that is mediated by donor immune cells. Antitumor activity is seen after non-myeloablative stem-cell transplantation in many patients, including those with CML, chronic lymphoblastic leukemia (CLL), acute leukemia, multiple myeloma, lymphoma and renal-cell carcinoma. A significant fraction of these patients, however, fail to respond or undergo relapse after an initial response . Additionally, GVHD occurs in approximately 50% of these patients and contributes to morbidity and mortality. These results demonstrate that the GVL effect can sometimes replace intensive chemoradiotherapy, but highlight the need for a clearer understanding of the immunological mechanisms and target molecules that are required for elimination of malignant cells. Such conditioning, together with administration of a TAC, may provide a means for augmenting or maintaining a GVL effect while providing a means of reducing or eliminating the cancer cells in the blood of a patient.

By appropriate formulation of the TAC (such as enterically coated capsules), it can be delivered to the entire mucosal surface of the intestine and/or the liver in high doses. Thus, the TAC can achieve high concentrations in the intestinal mucosa where the initiating alloimmune recognition event is taking place.

The method of the present invention employs oral administration of an effective amount of a TAC to a patient who has undergone or will undergo allogeneic hematopoietic cell or organ allograft transplantation. Representative TACs include, but are not limited to, beclomethasone 17,21-dipropionate, alclometasone dipropionate, budesonide, 22S budesonide, 22R budesonide, beclomethasone-17-monopropionate, clobetasol propionate, diflorasone diacetate, flunisolide, flurandrenolide, fluticasone propionate, halobetasol propionate, halcinocide, mometasone furoate, and triamcinalone acetonide. Such TACs are well known to those skilled in the field of, for example, intestinal disorders, and are commercially available from any number of sources. Suitable TACs useful in the practice of this invention are any that have the following characteristics: rapid first-pass metabolism in the intestine and liver, low systemic bioavailability, high topical activity, and rapid excretion (See Thiesen et al., Alimentary Pharmacology & Therapeutics 10:487-496 (1996)) (incorporated herein by reference).

In a preferred embodiment of this invention, the TAC is beclomethasone dipropionate (BDP). BDP has a chemical formula of C.sub.28H.sub.37ClO.sub.7, and is available from a number of commercial sources, such as Schering-Plough Corporation (Kenilworth, N.J.) or Pharmabios in Italy in bulk crystalline form. 33DP has the following structure:

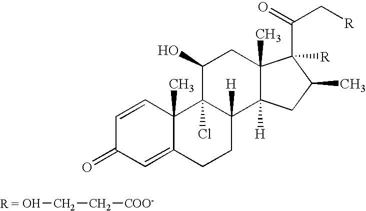

The TAC may be formulated for oral administration by techniques well known in the formulation field, including formulation as a capsule, pill, coated microsphere with specific dissolution qualities (i.e., a quick or slow-dissolving format), or emulsion. In the practice of this invention, at least two separate dosage forms of a TAC are administered to a patient in need thereof. The use of two different dosage forms allows the patient to receive TAG throughout the entire gastrointestinal tract, from the stomach to the rectum. It is preferable to limit the number of separate dosage forms to the smallest number possible; thus, two separate dosage forms is the preferred embodiment. The effective amount of TAC in each dosage form may vary from patient to patient, and may be readily determined by one skilled in the art by well-known dose-response studies. Such effective amounts will generally range between about 0.1 mg/day to about 8 mg/day, and more typically range from about 2 mg/day to about 4 mg/day. Accordingly, suitable capsules or pills generally contain from 1 rag to 2 mg TAC, and typically about 1 mg TAC, plus optional fillers, such as lactose, and may be coated with a variety of materials, such as cellulose acetate phthalate. By appropriate coating, such capsules, microspheres or pills may be made to dissolve within various location of the intestinal tract. For example, enteric-coated capsules prepared with a coating of cellulose acetate phthalate are known to dissolve in the alkaline environment of the small bowel, thus delivering its content to the small bowl and colon. Emulsions containing a TAC may also be employed for oral delivery, including optional emulsifying agents.

### EXAMPLES

The following examples are meant to be illustrative of the present invention and are not meant to be limited to such embodiments.

### Example I

### Control of GVHD by Treatment with BDP

A randomized, prospective, double blind, placebo controlled, multi-center pivotal trial was conducted to evaluate beclomethasone dipropionate (BDP) as a treatment of cancer by enhancing the graft versus leukemia effect (GVL) while controlling graft versus host disease (GVHD) following hematopoietic stem cell transplant in blood-borne cancer patients. The trial was divided into two phases, the purpose was to monitor short term effectiveness of BDP to control graft versus host disease in the first phase and the second phase was to assess the effect of the drug treatment on long term survival due to recurrence of hematologic malignancy or other causes of death.

To minimize the exposure of patients to long term exposure to systemic corticosteroids (prednisone or prednisolone), which are used as the standard of care to prevent or treat symptoms of graft versus host disease in conjunction with other immunosuppressive drugs, patients were treated with an oral formulation of BDP. BDP was formulated as two separate oral dosage forms an immediate release (IR) tablet and an enteric coated (EC) tablet. 129 patients were enrolled and 67 patients were randomized to placebo and 62 to BDP, Patients were at least 10 days post allogeneic hematopoietic cell transplantation, had gastrointestinal symptoms consistent with Grade II GVHD, and had endoscopic evidence of GVHD. The diagnosis of GVHD was confirmed by biopsy of the intestine (esophagus, stomach, small intestine, or colon) or skin. After being diagnosed with GVHD, patients were started on standard prednisone therapy. Patients were administered 2 mg/kg/day or 1 mg/kg/day for 10 days as a starting dose. After 10 days at this initial dose, prednisone was tapered over 7 days, after which the patients were maintained on a maintenance physiologic replacement dose of prednisone of 0.0625 mg/kg/day or 0.125 mg/kg/day. Concurrently, patients received BDP at a dose of 2 mg four times daily, for a total dose of 8 mg, or placebo for a maximum of 50 days.

Patients were monitored at clinic visits for evidence of increase in symptoms of GVHD (primary treatment failure). The primary efficacy endpoint was the time to treatment failure through study day 50. Treatment failure was defined as a worsening or recurrence of GVHD of such degree as to require an increase in immunosuppressive therapy. A subject was defined as a treatment failure if the patient required prednisone or equivalent IV corticosteroids at doses higher than that specified in the protocol, in response to uncontrolled signs or symptoms of GVHD; or required additional immunosuppressant medications other than those permitted by the protocol in response to uncontrolled signs or symptoms of GVHD. The time to treatment failure was calculated as the number of days elapsed between the randomization date and the date on which the subject was first identified as a treatment failure by the investigator.

Secondary efficacy endpoints included: 1) the time to treatment failure through study day 80, 2) the proportion of subjects who experienced treatment failure by study days 10, 30, 50, 60, and 80, and 3) Karnofsky performance status scores.

Safety was primarily assessed based on the following: 1) cumulative systemic corticosteroid exposure, 2) the incidence and degree of HPA axis suppression in patients who had not experienced treatment failure by study day 50, 3) rates of treatment-emergent adverse events, and 4) the overall survival rate 200 days post-transplant.

The primary analysis of the primary and secondary efficacy endpoints was based on the intent-to-treat principle. The analysis of the primary efficacy endpoint was based on the Kaplan-Meier method and log-rank test stratified by source of allograft. Hypothesis tests of the primary and secondary efficacy endpoints were performed using a two-sided significance level of 0.05. No adjustments were made to the significance level for inferential tests of the secondary efficacy endpoints. All patients who received at least one dose of BDP or placebo were included in the assessment of safety.

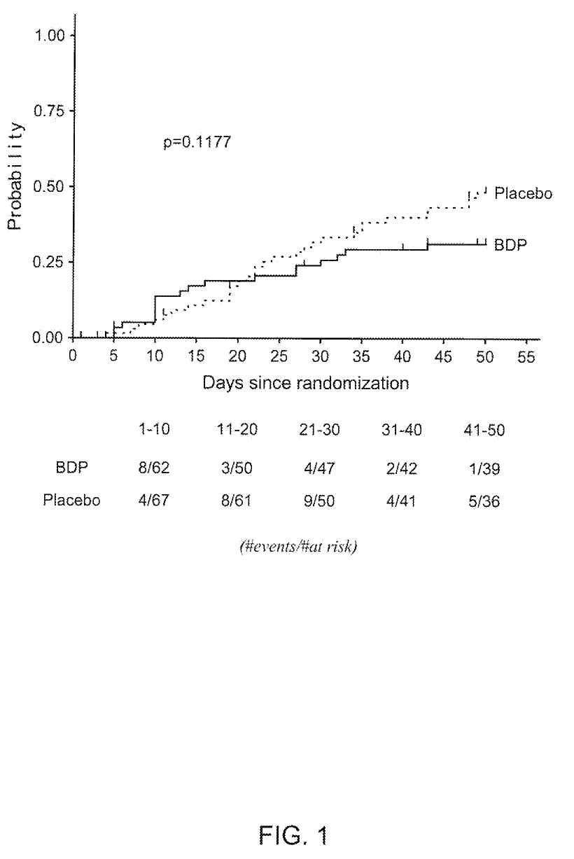

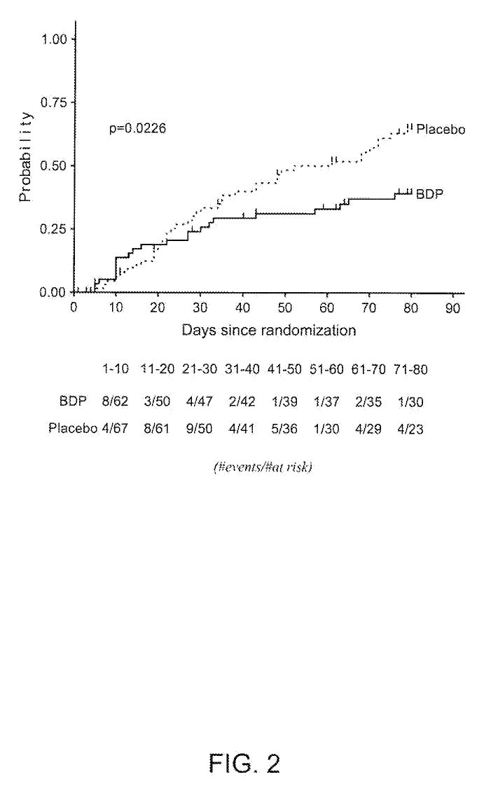

The primary efficacy endpoint of time to treatment, failure through study day 50 is summarized by “treatment group” in Table 1. Also summarized is the secondary endpoint of time to treatment failure through study day 80. Although these endpoints overlap, the latter endpoint includes events that occurred during the 30-day post-treatment, observation period and is intended to provide information on the durability of effect following treatment discontinuation. The Kaplan-Meier estimates for each endpoint are displayed in FIGS. 1 and 2, respectively.

As shown in FIG. 1, there was an initial increase in the treatment, failure rate for patients in the BDP group compared to placebo during the first 10 days of study treatment. Eight patients in the BDP group met the treatment failure endpoint during this period compared to 4 patients in the placebo group. Shortly after the start of the prednisone taper, approximately 10 days post-randomization, a difference between the BDP and placebo groups emerged (in favor of the BDP group) and steadily increased throughout the remainder of the 50-day study treatment period, such that by study day 50, the cumulative treatment failure rate was 31% for BDP versus 48% for placebo (p=0.0515, 2-test).

During the 50-day study treatment period, the risk of treatment failure was reduced by 37% for patients in the BDP group relative to placebo (hazard ratio 0.63; 95% CI: 0.35, 1.37); however, the primary inferential comparison for this endpoint was not statistically significant (p=0.1177, stratified log-rank test), This comparison includes all treatment failures observed during the 50-day study treatment period, including the 12 events that occurred during the first 10 days of treatment when all patients were receiving high-dose corticosteroids (1-2 mg/kg/day). It should be noted that 44% of the total number of treatment failures for BDP occurred within the first 10 days of randomization and prior to the prednisone taper. This compares to 13% of the treatment failures for placebo during this same period.

The time to treatment failure through study day 80 was also evaluated to assess the durability of response, and includes treatment failures that occurred during the 50-day study treatment period and 30-day post-treatment observation period. As shown in FIG. 2, the emerging difference between treatment groups that was observed during the 50-day treatment period continued to increase throughout the 30-day post-treatment observation period such that the overall cumulative treatment failure rate by study day 80 was 39% for BDP versus 65% for placebo (p=0.0048, Z-test).

For the entire 80-day study period, the risk of treatment failure was statistically significantly reduced by 44% for patients in the BDP group relative to placebo (hazard ratio 0.56; 95% CI: 0.33, 0.94; p=0.0226, stratified log-rank test). In addition to the decreased risk, the median time to treatment failure was increased by more than 28 days for the BDP group compared to placebo.

TABLE 1
Results of Intent-to-Treat Analysis of the Time to Treatment Failure
through Study Days 50 and 80 (All Randomized Subjects)

Treatment Group

| | Placebo | BDP | |
|---|---|---|---|
| Endpoint | N = 67 | N = 62 | P-value |
| Time to treatment | | | |
| failure through Study | | | |
| Day 50 | | | |
| Number with treatment | 30 | 18 | |
| failure | | | |
| Treatment failure rate | 0.48 (0.39, | 0.31 (0.23, | 0.0515 |
| by Study Day 50 | 0.60) | 0.43) | |
| Median time to | Not achieved | Not achieved | |
| treatment failure (95% | | | |
| CI) | | | |
| Hazard ratio (95% CI) | 0.63 (0.35, 1.37) | | 0.1177 |
| Time to treatment | | | |
| failure through Study | | | |
| Day 80 | | | |
| Number with treatment | 39 | 22 | |
| failure | | | |
| Treatment failure rate | 0.65 (0.55, | 0.39 (0.30, | 0.0048 |
| by Study Day 80 | 0.76) | 0.52) | |
| Median time to | 52 days (35, | Not achieved | |
| treatment failure (95% | 75) | | |
| CI) | | | |

| | | |
|---|---|---|
| Hazard ratio (95% CI) | 0.56 (0.33, 0.94) | 0.0226 |

The hazard ratio was estimated from a univariate Cox proportional hazards model. Placebo serves as the reference group.

### Example II

### Enhancement of the GVL Effect by Treatment with BDP

Treatment with BDP was associated with a statistically significantly higher overall survival rate 200 days post-transplant relative to placebo (p=0.006, Z-test). Based on Kaplan-Meier estimates, the overall survival rate 200 days post-transplant was 0.91 for the BDP group (95% CI: 0.66, 0.84) versus 0.74 for placebo (95% CI: 0.66, 0.84). The most common primary cause of death was relapse of the underlying malignancy, which occurred in 6 patients in the placebo group (9%) and in 2 patients in the BDP group (3%). The second most common cause of death appeared to be sepsis.

Based on a univariate time-dependent Cox proportional hazards model, the risk of mortality during this period was 68% lower following the initiation of treatment with BDP when compared to no treatment (hazard ratio 0.32; 95% CI: 0.12, 0.87; p=0.0252). A multivariate Cox model was used to evaluate the effect of BDP while simultaneously accounting for selected competing causes of mortality after hematopoietic cell transplant. The competing causes of mortality included the subject's age and gender, intensity of the conditioning regimen (myeloablative, non-myeloablative), primary diagnosis, transplant source (bone marrow, peripheral blood stem cells), and degree of HLA match, with greater benefit seen in the patients receiving non-myeloabaltive pre-conditioning. The results of the multivariate model are displayed in Table 2. With the exception BDP treatment (hazard ratio 0.32; 95% CI: 0.11, 0.89; p=0.0292), none of the factors included in the model were statistically significantly associated with the duration of survival during the 200-day period following transplant.

An exploratory analysis was also performed to evaluate the relationship between the treatment failure endpoint during the 80-day study period and duration of overall survival during the 200-day period following transplant. Based on a time-dependent Cox proportional hazards model, patients who experienced treatment failure during this period had a statistically significantly greater risk of death (due to any cause) during the 200-day post-transplant period relative to patients who did not experience treatment failure (hazard ratio 3.36; 95% CI: 1.36, 8.29; p=0.0085).

TABLE 2
Multivariate Proportional Hazards Model for the Duration of Overall
Survival 200 Days Post-Transplant (Safety Population)

| | Coefficient | HR | | P- |
|---|---|---|---|---|
| Variable | (bᵢ) | [exp(bᵢ)] | 95% CI | value |
| BDP | −1.155 | 0.32 | (0.11, | 0.0292 |
| Males | 0.225 | 1.25 | (0.52, | 0.6174 |
| Age (per 1-year | 0.016 | 1.02 | (0.98, | 0.3496 |
| Non-ablative | 0.207 | 1.23 | (0.48, | 0.6705 |
| 2 HLA haplotype | −0.758 | 0.47 | (0.20, | 0.0793 |
| Bone marrow as source | −0.722 | 0.49 | (0.06, | 0.4910 |
| Primary diagnosis | 0.290 | 1.34 | (0.48, | 0.5753 |
| associated with an | | | 3.69) | |
| elevated risk of | | | | |
| disease-related | | | | |
| mortality | | | | |

HR = hazard ratio; CI = confidence interval.

The hazard ratio for each variable was estimated from a multivariate Cox proportional hazards model.

This study shows an improvement in outcome for all parameters measured in patients with intestinal GVHD treated with oral BDP, While the primary efficacy variable (time to treatment failure, in the first 50 days post randomization) showed a clear trend towards efficacy, there was a clear-cut statistical and clinically meaningful advantage over the first 80 days. The improvement in time to treatment failure was accompanied by a 69% relative reduction in mortality at 200 days post transplant, the prospectively defined survival endpoint.

This study showed that patients treated with a 10-day induction course of prednisone followed by a rapid prednisone taper and oral BDP, 2 mg four times daily for 50 days, have an improved outcome compared to patients treated with the same prednisone induction plus placebo, as measured by proportion of treatment failures at various time points, time to treatment failure to study day 80, as well as survival at transplant day 200. These improvements in outcome are achieved without an increase in clinically significant toxicity, yielding a favorable risk to benefit ratio. A multivariable Cox proportional hazards model, taking into account competing risk factors for mortality, for the duration of survival at day-200 post transplant shows that randomization to oral BDP leads to significantly less mortality (hazard ratio 0.32, 95% confidence interval 0.11-0.89, p=0.029) and improved survival.

There was a significant correlation between both treatment failure and corticosteroid exposure and survival, demonstrated by the decrease in deaths in the treatment group due to both infection and relapse of underlying disease. This result is due to enhancement of the graft-versus-leukemia effect while diminishing the graft-vs-host reaction.

In addition to the TAC, acceptable carriers and/or diluents may be employed and are familiar to those skilled in the art. Formulations in the form of pills, capsules, microspheres, granules or tablets may contain, in addition to one or more TACs, diluents, dispersing and surface active agents, binders and lubricants. One skilled in the art may further formulate the TAC in an appropriate manner, and in accordance with accepted practices, such as those disclosed in Remington's Pharmaceutical Sciences, Gennaro, Ed., Mack Publishing Co., Easton, Pa., 1990 (incorporated herein by reference).

As optional components, other active agents may be administered in combination with the TAC, including (but not limited to) prednisone, prednisolone, cyclosporins, methotrexate, tacrolimus and biological agents that affect T-lymphocytes” such as anti-lymphocyte globulin, anti-T-cell monoclonal antibodies or anti-T-cell immunotoxins. Prednisone or prednisolone are preferably administered at a concentration of at least about 1 mg/kg body weight/day.

Various patents and publications are cited herein, and their disclosures are hereby incorporated by reference in their entireties. The present invention is not intended to be limited in scope by the specific embodiments described herein. Although the present invention has been described in detail for the purpose of illustration, various modifications of the invention as disclosed, in addition to those described herein, will become apparent to those of skill in the art from the foregoing description. Such modifications are intended to be encompassed within the scope of the present claims.

What is claimed is:

1. A method of treating cancer comprising the steps of:
(a) administering an effective amount of a topical active corticosteroid; and
(b) initiating a form of non-myeloablative conditioning, wherein the steps of (a) and (b) are sufficient to reduce or eliminate cancer cell levels in an individual.

2. The method of claim 1, wherein the topical active corticosteroid is beclomethasone 17,21-dipropionate.

3. The method of claim 2, wherein the beclomethasone 17,21-diproprionate is administered orally at a dosage of between about 0.1 mg per day to about 8 mg per day.

4. The method of claim 1, wherein the topical active corticosteroid is administered in combination with prednisone or prednisolone at a concentration of at least 1 mg/kg body weight/day.

5. The method of claim 1, wherein the topical active corticosteroid is formulated for oral administration in the form of a pill, tablet, capsule or microsphere.

6. The method of claim 1, wherein the non-myeloablative conditioning comprises administration of an agent selected from the group consisting of fludarabine, busulfan, ATG and melphalan.

7. A method of treating cancer by maintaining or augmenting a graft-versus-leukemia effect in an individual comprising the steps of administering an effective amount of a topical active corticosteroid and initiating a form of non-myeloablative conditioning, wherein the levels of cancer cells in the individual are reduced or eliminated.

8. The method of claim 7, wherein the topical active corticosteroid is beclomethasone 17,21-dipropionate.

9. The method of claim 8, wherein the beclomethasone 17,21-diproprionate is administered orally at a dosage of between about 0.1 mg per day to about 8 mg per day.

10. The method of claim 7, wherein the topical active corticosteroid is administered in combination with prednisone or prednisolone at a concentration of at least 1 mg/kg body weight/day.

11. The method of claim 7, wherein the topical active corticosteroid is formulated for oral administration in the form of a pill, tablet, capsule or microsphere.

12. The method of claim 7, wherein the non-myeloablative conditioning comprises administration of an agent selected from the group consisting of fludarabine, busulfan, ATG and melphalan.
